(19) United States
(12) Patent Application Publication
(10) Pub. No.: US 2010/0011451 A1
(43) Pub. Date: Jan. 14, 2010

(54) PHENOTYPIC AND GENOTYPIC DIFFERENCES OF MVA STRAINS

(76) Inventor: Paul Chaplin, Munich (DE)

Correspondence Address: LAW OFFICE OF SALVATORE ARRIGO, 1050 CONNECTICUT AVE. NW, 10TH FLOOR, WASHINGTON, DC, 20036, US

(21) Appl. No.: 12/439,404

(22) PCT Filed: Sep. 6, 2007
(86) PCT No.: PCT/EP2007/007789

§ 371 Date: Feb. 27, 2009

Related U.S. Application Data
(60) Provisional application No. 60/843,001, filed on Sep. 8, 2006.

(51) Int. Cl. A61K 49/00; C12Q 1/68; A61P 43/00

(52) U.S. Cl. 800/3; 435/6

(57) ABSTRACT

The present invention provides kits and methods to screen viral nucleic acids for a profile of genetic deletions and mutations, optionally in combination with one or more assays for viral replication and/or attenuation capacity.

### FIELD OF THE INVENTION

The present invention provides methods and kits to screen viral nucleic acids for genetic deletions and mutations. The invention can be used in combination with viral replication and attenuation assays.

### BACKGROUND OF THE INVENTION

During the 1970's the pioneering work of Mayr and associates led to the development of safer vaccines against poxvirus infections (18, 19). This was achieved by continually passaging the chorioallantois vaccinia virus (CVA) on chicken embryo fibroblast (CEF) cells; after more than 570 such passages, the virus was re-named “Modified Vaccinia Ankara” (MVA) virus (11, 12). Reportedly, the safety and immunogenicity of this virus has been tested extensively and both the limited ability to replicate as well as the neuropathogenicity of MVA in humans and other mammals has been described in various publications (1, 11, 12, 13, 14, 18). Based on these reports, it has been generally concluded that after the 570th passage on CEF cells MVA is uniform and genetically stable (11), an assertion that reportedly is widely accepted today (15, 22).

These conclusions were supported by DNA mapping of MVA and its ancestor CVA by enzyme digests, which revealed six deletions within the MVA genome resulting in an estimated loss of 30 kb of DNA compared to its ancestor CVA (2, 14). The nucleotide sequence of MVA has been determined, the genes annotated and compared to the Vaccinia Copenhagen strain (3). The MVA genome, which has been computed to be 177 kb, allowed a more detailed analysis of deleted and altered genes. These data revealed the absence of some mammalian host range genes in MVA, which was taken as direct evidence for the limited replication in mammalian cells (3).

However, while certain studies have indicated that MVA fails to replicate in human cells (5, 12, 14, 21) others have clearly demonstrated that MVA does have a limited ability to replicate in various human cell lines, such as HeLa (4, 7, 26), 293, (7) and HaCat (6).

Due to such conflicting data, the importance of resolving this issue is one of patient safety through vaccination programs, and thus a need exists for new methods and kits for screening heterogeneous viral populations for the presence of first-indicators or markers of their attenuation and/or replication capacity.

### SUMMARY OF THE INVENTION

The invention encompasses methods of screening an MVA nucleic acid sample for mutations. In one embodiment, an MVA nucleic acid sample is prepared and it is determined whether the MVA nucleic acid sample includes one or more minority viral genotypes that have a different genomic DNA sequence than that of an MVA virus strain having at least one of the following properties: i) capability of reproductive replication in vitro in chicken embryo fibroblasts (CEF) but no capability of reproductive replication in the human keratinocyte cell line (HaCaT), the human embryo kidney cell line (293), the human bone osteosarcoma cell line (143B), and the human cervix adenocarcinoma cell line (HeLa), and (ii) failure to replicate in a mouse model that is incapable of producing mature B and T cells and as such is severely immune compromised and highly susceptible to a replicating virus.

In a further embodiment, said MVA virus strain has both of the advantageous properties.

Furthermore, MVA virus strains having the above-mentioned replication properties may also induce at least the same level of specific immune response in vaccinia virus prime/vaccinia virus boost regimes when compared to DNA-prime/vaccinia virus boost regimes.

An MVA virus strain having at least one and/or both of the aforementioned replication properties is hereinafter also denoted as “reference MVA virus strain”.

A particular strain having the aforementioned replication properties was deposited on Aug. 30, 2000 at the European Collection of Cell Cultures (ECACC) under number V00083008. This strain is referred to as “MVA-BN” throughout the Specification.

In a preferred embodiment, the sequence of the MVA nucleic acid differs from the DNA sequence of a reference MVA virus strain at one or more sites selected from deletion I site; nt 85017; nts 137398-404; nt 133176; nt 27698; nt 27699; nt 86576; nt 126375; nt 135664; nt 149358; and nt 153212 of said reference MVA virus strain. In a particular preferred embodiment, said reference MVA virus strain is MVA-BN.

In a preferred embodiment, the nucleic acid sample is analyzed by PCR to determine whether the MVA nucleic acid sample includes one or more minority viral genotypes having a different genomic DNA sequence than that of a reference MVA virus strain, such as, e.g., MVA-BN. In one embodiment, the MVA nucleic acid sample is prepared from an animal host. Preferably, the animal host is an immunocompromised mouse.

The invention also encompasses kits for screening an MVA nucleic acid sample for mutations. The kits can contain one or more oligonucleotide primers for amplifying an MVA nucleic acid by PCR. Preferably, said one or more primers amplify a segment of MVA DNA comprising one or more sites selected from deletion I site; nt 85017; nts 137398-404; nt 133176; nt 27698; nt 27699; nt 86576; nt 126375; nt 135664; nt 149358; and nt 153212 of a reference MVA virus strain, such as, e.g., MVA-BN.

### BRIEF DESCRIPTION OF THE DRAWINGS

The invention is more fully understood through reference to the drawings.

FIG. 1: The location of the six known deletion sites in the genome of MVA is graphed using the published lettering system.

FIG. 2: MVA viruses differ in their ability to replicate in vitro. Attenuation profiles of different poxviruses tested on the various cell lines listed were compiled as described in Example 1. A representative example (geometric mean and standard error) of three separate experiments is shown for each viral/cell combination.

FIG. 3: MVA differ in their ability to replicate in immune deficient mice. Survival of AGR129 mice after inoculation with various poxviruses was recorded as further described in Example 1. Immune deficient AGR129 mice were inoculated with 1×10⁷ TCID₅₀ of different poxviruses and survival was monitored. All animals were sacrificed 100 days after infection. Mean survival and standard error of three to 50 animals are illustrated.

FIG. 4: Deletion-profiling by PCR analysis of six proposed MVA deletion sites within various viruses, as further described in Example 1. DNA extracted from the different vaccinia viruses was amplified by PCR using primers (Table 1) flanking the deletion sites that have been mapped for MVA; the PCR products were size-fractionated on agarose gels. Representative examples of three to four separate experiments are shown in panel A (deletion site 1), panel B (deletion site II), panel C (deletion site III), panel D (deletion site IV), panel E (deletion site V) and panel F (deletion site VI).

FIG. 5. Deletion-profiling by PCR amplification and analysis of CVA-specific regions in various viruses, as further described in Example 1. DNA of the different vaccinia viruses were amplified by PCR using primers (Table 1) designed to amplify and detect the CVA loci that reportedly had been deleted within MVA; the PCR products were size-fractionated on agarose gels. Representative examples of three to four separate experiments are shown in panel A (CVA locus I), panel B (CVA locus II), panel C(CVA locus III), panel D (CVA locus IV), panel E (CVA locus V) and panel F (CVA locus VI).

### DESCRIPTION OF THE EMBODIMENTS

Reference will now be made in detail to the exemplary embodiments of the invention.

Modified Vaccinia Ankara (MVA) virus was originally developed by serial passages on chicken embryo fibroblast cells. After passage 570 the virus was considered homogenous, genetically stable and has been used extensively in a smallpox vaccination regime.

Three commonly used MVA virus strains (MVA 572, MVA-I721 and MVA-BN), previously reported as genetically stable, have been analyzed herein and shown by Polymerase Chain Reaction (PCR)-methods to contain six deletions within the genome characterized for MVA. MVA-572 (ECACC V94012707) was kindly provided by Prof. A. Mayr, Veterinary Faculty, University of Munich. MVA-I721 (CNCM 1721) was obtained by the Collection Nationale de Cultures de Microorganismes, Institut Pasteur (CNCM).

In the context of the present invention, “MVA virus strains” or “reference MVA virus strains” also refer to recombinant viruses derived therefrom. Methods to construct such recombinant viruses are known to a person skilled in the art.

All known vaccinia strains show at least some replication in the cell line HaCaT, whereas the reference MVA virus strains of the invention, in particular MVA-BN, do not reproductively replicate in HaCaT cells. In particular, MVA-BN exhibits an amplification ratio of 0.05 to 0.2 in the human embryo kidney cell line 293 (ECACC No. 85120602). In the human bone osteosarcoma cell line 143B (ECACC No. 91112502), the ratio is in the range of 0.0 to 0.6. For the human cervix adenocarcinoma cell line HeLa (ATCC No. CCL-2) and the human keratinocyte cell line HaCaT (Boukamp et al. 1988, J Cell Biol 106(3): 761-71), the amplification ratio is in the range of 0.04 to 0.8 and of 0.02 to 0.8, respectively. MVA-BN has an amplification ratio of 0.01 to 0.06 in African green monkey kidney cells (CV1: ATCC No. CCL-70). Thus, MVA-BN, which is a representative strain of the invention, does not reproductively replicate in any of the human cell lines tested.

The amplification ratio of a reference MVA virus strain, such as MVA-BN is clearly above 1 in chicken embryo fibroblasts (CEF: primary cultures). A ratio of more than “1” indicates reproductive replication since the amount of virus produced from the infected cells is increased compared to the amount of virus that was used to infect the cells. Therefore, the virus can be easily propagated and amplified in CEF primary cultures with a ratio above 500.

In a further preferred embodiment, the reference MVA virus strains of the invention, in particular MVA-BN, are characterized by a failure to replicate in vivo. In the context of the present invention, “failure to replicate in vivo” refers to viruses that do not replicate in humans and in the mouse model described below.

The “failure to replicate in vivo” can be preferably determined in a suitable mouse model. In the context of the present invention, it is imperative for a suitable mouse model to fulfill the requirement that the respective mice are incapable of producing mature B and T cells. In case said requirement is not fulfilled, the mouse model does not constitute a suitable model for demonstrating a “failure to replicate in vivo” according to the present invention. An example of such mice is the transgenic mouse model AGR129 (obtained from Mark Suter, Institute of Virology, University of Zürich, Zürich, Switzerland). This mouse strain has targeted gene disruptions in the IFN receptor type I (IFN-α/β) and type II (IFN-α/β) and type II (IFN-γ) genes, and in RAG. Due to these disruptions, the mice have no IFN system and are incapable of producing mature B and T cells, and as such, are severely immune-compromised and highly susceptible to a replicating virus. In addition to the AGR129 mice, any other mouse strain can be used that fulfills the requirement of being incapable of producing mature B and T cells, and as such, is severely immune-compromised and highly susceptible to a replicating virus. In particular, the viruses of the present invention do not kill AGR129 mice within a time period of at least 45 days, more preferably within at least 60 days, and most preferably within 90 days post infection of the mice with 10⁷ pfu virus administered via intra-peritoneal injection. Preferably, the viruses that exhibit “failure to replicate in vivo” are further characterized in that no virus can be recovered from organs or tissues of the AGR129 mice 45 days, preferably 60 days, and most preferably 90 days after infection of the mice with 10⁷ pfu virus administered via intra-peritoneal injection. Detailed information regarding the infection assays using AGR129 mice and the assays used to determine whether virus can be recovered from organs and tissues of infected mice can be found in the example section.

Furthermore, a reference MVA virus strain may also induce at least the same level of specific immune response in vaccinia virus prime/vaccinia virus boost regimes when compared to DNA-prime/vaccinia virus boost regimes. A vaccinia virus is regarded as inducing at least substantially the same level of immunity in vaccinia virus prime/vaccinia virus boost regimes if, when compared to DNA-prime/vaccinia virus boost regimes, the CTL response, as measured in one of the following two assays (“assay 1” and “assay 2”), preferably in both assays, is at least substantially the same in vaccinia virus prime/vaccinia virus boost regimes when compared to DNA-prime/vaccinia virus boost regimes. More preferably, the CTL response after vaccinia virus prime/vaccinia virus boost administration is higher in at least one of the assays, when compared to DNA-prime/vaccinia virus boost regimes. Most preferably, the CTL response is higher in both of the following assays.

Assay 1: For vaccinia virus prime/vaccinia virus boost administrations, 6-8 week old BALB/c (H-2d) mice are prime-immunized by intravenous administration with 10⁷ TCID₅₀ vaccinia virus of the invention expressing the murine polytope as described in Thomson et al., 1998, J. Immunol. 160, 1717 and then boost-immunized with the same amount of the same virus, administered in the same manner three weeks later. To this end, it is necessary to construct a recombinant vaccinia virus expressing the polytope. Methods to construct such recombinant viruses are known to a person skilled in the art and are described in more detail below. In DNA prime/vaccinia virus boost regimes the prime vaccination is done by intra muscular injection of the mice with 50 μg DNA expressing the same antigen as the vaccinia virus. The boost administration with the vaccinia virus is done in exactly the same way as for the vaccinia virus prime/vaccinia virus boost administration. The DNA plasmid expressing the polytope is also described in the publication referenced above, i.e., Thomson, et al. In both regimes, the development of a CTL response against the epitopes SYI, RPQ and/or YPH is determined two weeks after the boost administration. The determination of the CTL response is preferably done using the ELISPOT analysis as described by Schneider, et al., 1998, Nat. Med. 4, 397-402. The viruses of the invention are characterized in this experiment in that the CTL immune response against the epitopes mentioned above, which is induced by the vaccinia virus prime/vaccinia virus boost administration, is substantially the same, preferably at least the same, as that induced by DNA prime/vaccinia virus boost administration, as assessed by the number of IFN-γ producing cells/10⁶ spleen cells.

Assay 2: This assay basically corresponds to assay 1. However, instead of using 10⁷ TCID₅₀ vaccinia virus administered i.v., as in Assay 1; in Assay 2, 10⁸ TCID₅₀ vaccinia virus of the present invention is administered by subcutaneous injection for both prime and boost immunization. The virus of the present invention is characterized in this experiment in that the CTL immune response against the epitopes mentioned above, which is induced by the vaccinia virus prime/vaccinia virus boost administration, is substantially the same, preferably at least the same, as that induced by DNA prime/vaccinia virus boost administration, as assessed by the number of IFN-y producing cells/10⁶ spleen cells.

The strength of a CTL response as measured in one of the assays shown above corresponds to the level of protection.

In one embodiment, the methods of the invention allow the identification of populations of MVA viruses, such as among those commercially available or those deposited in viral libraries, as complex polyclonal mixtures of vaccinia viruses, the composition of which appears to govern their growth in human cells. Without being bound by one theory, these phenotypic properties of MVA can be altered by passaging and/or limiting dilution (part of an amplification process), presumably by changing the composition and/or by additional mutations of the viruses within MVA. Thus, the invention provides new methods of profiling viral populations for newly-identified mutations and deletion patterns that serve as a first round screening of attenuation-deficient variants.

The invention provides methods to screen viral populations for molecular indicators of their attenuation and/or replication potential. In one embodiment, one or more MVA viruses are compared in terms of their ability to replicate in human cells (a measure of replication potential) and/or their safety in immune compromised mice (a measure of attenuation). In another embodiment, the in vivo and/or in vitro analysis of the viruses' attenuation/replication potential is combined with an analysis of the viral genomes by PCR. In yet another embodiment, viral sequencing is additionally included in the screening method. The sequencing-based screening can be directed to specific regions of the genome containing one or more of the mutations identified in Table 3. In another embodiment, the sequencing-based screening is directed to the regions other than those containing the mutations in Table 3. In yet another embodiment, both the regions containing the mutations summarized in Table 3 and other regions are both sequenced.

In one embodiment, one, two, three or more MVA viruses are evaluated that belong to the group comprising MVA-572, a plaque purified MVA, which was used as a combination smallpox vaccine in conjugation with a vaccinia virus in more than 120,000 people during the late 1970's (18, 19); MVA-I721 was reportedly created by passaging an MVA strain obtained from Mayr (MVA 570,11) in CEF cells (1) and MVA-BN that was obtained by limiting dilution and further passaging of MVA-572 in CEF cells (6, 20).

In one embodiment, one or more of the viruses to be screened can show a limited ability to replicate in various human cell lines, such as HeLa (4, 7, 26), 293, (7) and HaCat (6). In one embodiment, two or more publicly deposited MVA viruses are compared in terms of their ability to replicate in human cells and their safety in immune compromised mice, followed by an analysis of their genomes by PCR and sequencing using the methods of the invention. In another embodiment, one or more MVA viruses are first amplified in vivo or in vitro using the methods of the invention, from one or more populations of deposited viruses, such as MVA-572, MVA-I721 and MVA-BN, and the amplified viral population obtained by limiting dilution and further passaging in CEF cells (6, 20). In another embodiment, the amplification is done in vivo, for example using immune-compromised mice such as AGR129.

In one embodiment, MVA-I721 and MVA-572 are shown to differ from MVA-BN in that they both have the ability to replicate in one or more human cell line(s) and in immune deficient mice (AGR129 strain), In one embodiment, the results of the molecular screening correlates with a phenotypic analysis of the viral composition that indicates the presence of viral variants that can be isolated from the AGR129 mice inoculated with the deposited virus, such as in MVA-572 or MVA-I721. In one embodiment, the phenotypic analysis in terms of viral composition (e.g., monoclonal, polyclonal) correlates with different phenotypes as measured in assays for in vitro and/or in vivo growth/replication potential relative to that of MVA-BN. The term “viral variants” denotes viruses differing from a reference MVA virus strain, such as MVA-BN in that they have, e.g., the ability to replicate in human cells and/or in immune deficient mice such as the AGR129 strain. Said “viral variants” may correspond to minority viral genotypes of the respective inoculated virus. In the context of the present invention, a “minority viral genotype” comprises a minor fraction of the total MVA virus population inoculated in, e.g., an immune deficient mouse. Such a minority viral genotype may display other properties than the major fraction of inoculated MVA virus. Furthermore, the total MVA virus population including minority viral genotypes may also have different properties compared to a reference MVA virus strain, such as, e.g., MVA-BN.

In one embodiment, the viral variants do not contain in their genome all the six deletions characterized for MVA. In another embodiment, the variants differ in nucleotide composition from the published sequence (3). In some embodiments, the variants are identified in DNA prepared from bulk viral preparations of MVA-572, MVA-I721 and MVA-BN that share a 100% identical nucleotide sequence in their coding regions, indicating that the fraction of the viral variant relative to the bulk of the virus is such that the majority of the viruses first appear to be the same viruses.

In another embodiment, the screening methods of the invention allow the determination whether or not MVA viruses, such as those deposited in viral banks and publicly available as MVA-572 and MVA-I721 contain a mixture of vaccinia viruses, some of which with undesirable properties. Non-limiting examples of such undesirable properties include in vitro replication potential in one or more type of mammalian cell (e.g., human cell, mouse cell) and replication potential in one or more types of mammalian cell in an organism (e.g., in a human, in a mouse) whose immune system may be or may not be compromised. In one embodiment, the presence of vaccinia viruses with undesirable properties is not detectable in a given viral population by nucleotide sequence without a prior amplification step (in vitro and/or in vivo) of either the virus(es) or its/their DNA that is part of the polyclonal mixture; since nucleotide sequencing of any given viral population may typically yield the sequence of the predominant viral genome within a polyclonal mixture of vaccinia viruses. In one embodiment, the virus whose sequence contains one or more of the mutations identified in this invention (e.g., those listed in Table 3), is a virus representing a minority viral genotype whose DNA sequence is only found to be present in a given viral population (e.g., one of those deposited in cell and viral banks) after that given viral population is amplified in vitro or in vivo. In one embodiment, the in vitro amplification of the virus is done in a human cell line. In another embodiment, the amplification in a human cell line is combined with an amplification in a mammalian organism or in a mammalian organ, such as in mouse or in mouse ovaries.

Deletions in the viral genome can be detected by, for example, molecular assays such as polymerase chain reaction (PCR), primer extension, restriction fragment length polymorphism, in situ hybridization, reverse transcription-PCR, and differential display of RNA. A particularly preferred method of deletion detection includes a polymerase chain reaction. PCR methods are known in the art, as are guidelines for choosing specific primers to detect specific deletions. The PCR method can involve touch-down PCT, real-time PCR, fluorescence-based PCR sequencing, or a combination of other methods of PCR-based DNA amplification and/or sequencing. In one embodiment, the primers selected include one or more of the primers listed in Table 1.

One of skill in the art would understand that details of reaction protocols and parameters considered in choosing appropriate primers are found in standard references such as Sambrook et al., Molecular Cloning: A Laboratory Manual, Cold Spring Harbor Laboratory, 3^{rd} Edition, 2001; Dieffenbach and Dveksler, PCR Primer: A Laboratory Manual, Cold Spring Harbor Laboratory, 2^{nd}, 2003; PCR Protocols: Current Methods and Applications (Methods in Molecular Biology, 15) by Bruce A. White (Ed.), 1993; and in the examples below. The practice of the invention employs additional techniques of molecular biology, protein analysis and microbiology, which are within the knowledge of the skilled practitioner of the art. Such techniques are explained fully in, for example, Current Protocols in Molecular Biology, Ausubel et al., eds., John Wiley & Sons, New York, 1995. Also, the techniques for transfection and infection of the cells, amplification and titration of the viruses have been described previously. For example, see F. L. Graham et al., Molecular Biotechnology 3: 207-220 (1995); Crouzet et al., Proc. Natl. Acad. Sci. USA 94: 1414-1419 (1997); U.S. Pat. Nos. 6,761,893 and 6,913,752 and European Patent No. 1 335 987, all of which are herein specifically incorporated by reference.

### Methods and Kits for Screening for Mutations

The invention encompasses methods for screening an MVA nucleic acid sample for mutations, comprising the determination if said nucleic acid sample includes minority viral genotypes having a different genomic DNA sequence than that of a reference MVA virus strain. These mutations can be associated with the replicative ability of the MVA virus in certain cell types, for example human cells, and in animal hosts. The mutations include those described in Table 3.

In a preferred embodiment, the DNA sequence of an MVA nucleic acid sample including one or more minority viral genotypes differs from the DNA sequence of a reference MVA virus strain at a site selected from deletion I site; nt 85017; nts 137398-404; nt 133176; nt 27698; nt 27699; nt 86576; nt 126375; nt 135664; nt 149358; and nt 153212 of a reference MVA virus strain. The sequences may vary at one or more of these sites. In a particular preferred embodiment, said reference MVA virus strain is MVA-BN.

In one embodiment the MVA nucleic acid sample including one or more minority viral genotypes differs from the DNA sequence of MVA-BN at deletion I site; nt 85017; nts 137398-404; and nt 133176. In another embodiment, the MVA nucleic acid sample including one or more minority viral genotypes differs from the DNA sequence of MVA-BN at 137398-404; nt 133176; nt 27698; nt 27699; nt 86576; nt 126375; nt 135664; nt 149358; and nt 153212.

The existence of differences between the MVA nucleic acid sample including one or more minority viral genotypes and the DNA sequence of a reference MVA virus strain, such as, e.g., MVA-BN, can be determined by many techniques known in the art. For, example, DNA sequencing of cloned DNAs or amplified DNA fragments, such as PCR products, can be used to identify differences in nucleic acid sequence. In another embodiment, probes are used that can hybridize preferentially or exclusively to a sequence containing either the MVA-BN or mutant sequence. In a further embodiment, restriction enzyme digestion is used to differentiate the mutant and MVA-BN sequences due to alteration or creation of a restriction site by a mutation.

Preferably, one or more PCR primers spanning a site selected from deletion I site; nt 85017; nts 137398-404; nt 133176; nt 27698; nt 27699; nt 86576; nt 126375; nt 135664; nt 149358; and nt 153212 of MVA-BN are used to amplify these sites of the MVA nucleic acid sample including one or more minority viral genotypes to determine the presence of mutations at these sites relative to the DNA sequence of MVA-BN. Differences between the sequence of the amplified fragment and the sequence of MVA-BN are determined by sequencing or using a specific probe.

In one embodiment, the presence of a difference between the MVA nucleic acid sample including one or more minority viral genotypes and the DNA sequence of MVA-BN is correlated with the ability of a virus containing mutated MVA nucleic acid sequence to replicate in a certain cell type, for example human cells, such as HeLa or HaCat, or in an animal host.

The invention also encompasses kits for screening an MVA nucleic acid sample for mutations. These mutations can be associated with the replicative ability of the MVA virus in certain cell types, for example human cells, and in animal hosts. The mutations include, but are not limited to, those described in Table 3.

In a preferred embodiment, the present invention relates to a method of screening an MVA nucleic acid sample for mutations comprising: preparing an MVA nucleic acid sample; and determining whether the MVA nucleic acid sample includes minority viral genotypes having a different genomic DNA sequence at a site selected from one or more of the following sites: deletion I site; nt 85017; nts 137398-404; nt 133176; nt 27698; nt 27699; nt 86576; nt 126375; nt 135664; nt 149358; and nt 153212 of an MVA having at least one of the following properties: i) capability of reproductive replication in vitro in chicken embryo fibroblasts (CEF) but no capability of reproductive replication in the human keratinocyte cell line (HaCaT), the human embryo kidney cell line (293), the human bone osteosarcoma cell line (143B), and the human cervix adenocarcinoma cell line (HeLa), and (ii) failure to replicate in a mouse model that is incapable of producing mature B and T cells and as such is severely immune compromised and highly susceptible to a replicating virus.

In a preferred embodiment, said MVA has both of properties (i) and (ii).

In a particular embodiment of the invention, said MVA is MVA-BN as deposited on Aug. 30, 2000 at the European Collection of Cell Cultures (ECACC) under number V00083008.

In a preferred embodiment, the kit contains one or more probe(s) capable of detecting the presence or absence of a mutation. In one embodiment, the kit contains one or more oligonucleotide primer(s) for amplifying, for example by PCR, a specific site of MVA-BN containing a mutation. Preferably, said one or more primers amplify a segment of MVA DNA comprising a site selected from deletion I site; nt 85017; nts 137398-404; nt 133176; nt 27698; nt 27699; nt 86576; nt 126375; nt 135664; nt 149358; and nt 153212 of MVA-BN.

In one embodiment, the term “kit” refers to components packaged or marked for use together and/or for sale together. For example, a kit can contain one or more sets of primers, a carrier, a DNA sample to serve as a positive control, a DNA sample to serve as a negative control; the components can be in one or more separate containers. In another example, a kit can contain any two components in one container, and a third component and any additional components in one or more separate containers. Optionally, a kit further contains instructions for combining and/or administering the components so as to formulate or be a part of a composition (a reaction mixture) suitable for testing (e.g., assessing replication potential, assessing patient safety, assessing attenuation, profiling a viral population) a viral sample for the presence of minority viral genotypes exhibiting one or more mutations as described in Table 3.

The description herein is put forth to provide those of ordinary skill in the art with a complete disclosure of how to make and how to use the present invention, and is not intended to limit the scope of what the inventors regard as their invention, nor is it intended to represent that the experiments set forth are all or the only experiments performed.

While the present invention is described with reference to the specific embodiments thereof, it should be understood by those skilled in the art that various changes may be made and equivalents may be substituted without departing from the true spirit and scope of the invention. In addition, many modifications can be made to adapt to a particular situation, material, composition of matter, process, process step or steps, to the objective, spirit, and scope of the present invention. All such modifications are intended to be within the scope of the claims appended hereto.

The specification is most thoroughly understood in light of the cited references, all of which are hereby incorporated by reference in their entireties.

Additional objects and advantages of the invention will be set forth in part in the description, which follows, and in part will be obvious from the description, or may be learned by practice of the invention. The objects and advantages of the invention will be realized and attained by means of the elements and combinations particularly pointed out in the appended claims. Moreover, advantages described in the body of the specification, if not included in the claims, are not, per se, limitations to the claimed invention.

Unless defined otherwise, the meanings of all technical and scientific terms used herein are those commonly understood by one of ordinary skill in the art to which this invention belongs.

It must be noted that, as used herein and in the appended claims, the singular forms “a,” “or,” and “the” include plural referents unless the context clearly dictates otherwise. Thus, for example, reference to “a subject polypeptide” includes a plurality of such polypeptides and reference to “the agent” includes reference to one or more agents and equivalents thereof known to those skilled in the art, and so forth.

Further, all numbers expressing quantities of ingredients, reaction conditions, % purity, polypeptide and polynucleotide lengths, and so forth, used in the specification, are modified by the term “about,” unless otherwise indicated. Accordingly, the numerical parameters set forth in the specification and claims are approximations that may vary depending upon the desired properties of the present invention. At the very least, and not as an attempt to limit the application of the doctrine of equivalents, each numerical parameter should at least be construed in light of the number of reported significant digits, applying ordinary rounding techniques. Nonetheless, the numerical values set forth in the specific examples are reported as precisely as possible. Any numerical value, however, inherently contains certain errors from the standard deviation of its experimental measurement.

### EXAMPLES

### Example 1

### Attenuation and Genetic Deletion Profiling of Various MVA Viruses

1.1. Animals, Cells and Viruses

AGR129 mice have the genes for the interferon (IFN) receptors I and II deleted, as well as recombination activating gene (RAG). As such, the mice have no functional natural killer (NK) cells, macrophages or mature T and B cells (9, 23).

The human cell lines HeLa (cervix carcinoma cell line, ECACC No. 93021013), TK-143B (bone osteosarcoma cell line, ECACC No. 91112502) and 293B (human embryo kidney epithelial cell line, ECACC No. 85120602) were obtained from the European Collection of Animal Cell Cultures (ECACC). The human keratinocyte cell line HaCat was obtained from the German Cancer Research Centre (DKFZ, Heidelberg, Germany). The murine dendritic like cell line AG101 was used as described (16). Primary CEF cells were prepared from 10-12 day old chicken embryos (21) derived from Specific Pathogen Free hen eggs (Charles River, Mass., USA). The cells were maintained in a humidified 5% CO₂ atmosphere incubator at 37° C. Primary CEF cells were grown in RPMI-1640 medium (Invitrogen, Karlsruhe, Germany). All other cell lines were grown in Dulbecco's Modified Eagle's Medium (DMEM; Invitrogen, Karlsruhe, Germany) supplemented with 10% fetal calf serum (FCS; PAA, Coelbe, Germany). The human cell line 143B was additionally supplemented with 15 μg/ml of 5-bromo-2′-deoxyuridine (Sigma-Aldrich, Munich, Germany).

Viruses used for this study were CVA and several isolates of MVA. CVA, MVA-572 (ECACC V94012707), Vaccinia Lister-Elstree (Elstree) and Vaccinia Western Reserve (WR) were kindly provided by Prof. A. Mayr, Veterinary Faculty, University of Munich. Vaccinia New York City Board of Health (NYCBH, ATCC VR-325) was obtained by the American Type Culture Collection (ATCC). MVA-I721 (CNCM 1721) was obtained by the Collection Nationale de Cultures de Microorganismes, Institut Pasteur (CNCM). MVA-BN was deposited at ECACC (V00083008). MVA isolated from AGR129 mouse ovaries (AGR-MVA-I721, AGR-MVA-572pre and AGR-MVA-572seq) are described within the text.

Additional details of methods used to manipulate the cells, animals and viruses according to the invention are described in U.S. Pat. Nos. 6,761,893 and 6,913,752 and the European Patent No. 1 335 987, all of which are herein specifically incorporated by reference.

1.2. Virus Titration

The titration was performed in a TCID₅₀-based assay on CEF cells in 96-well plates using 3 replicates for each triplicate viral sample (see above). Two to three day old CEF cells were seeded at 1×10⁵ cells/ml in 96-well plates (100 μl/well) and incubated at 37° C., 5% CO₂ overnight. Following incubation, the viral, positive (an MVA standard of known titer) and negative (media alone) control samples were diluted in RPMI-1640 from 10-1-10-10 and added to the 96-well plates (100 μl/well). Plates were incubated for a further 5 days at 37° C., 5% CO₂. The virus/media suspensions were then discarded and the cells fixed by the addition of 100 μl/well acetone/methanol solution (Merck, Darmstadt, Germany/NeoLab, Heidelberg, Germany). The acetone/methanol solution was then discarded and the plates allowed to dry. Plates were washed once with PBS-Tween 20 (PBS-T, 0.05% v/v) and incubated with 100 μl/well (1:1000 dilution) of a rabbit anti-vaccinia polyclonal immunoglobulin G (IgG) antibody (Quartett, Berlin, Germany) for 1 h at room temperature (RT). The cells were again washed two times with PBS-T, and then incubated with 100 μl/well anti-rabbit-IgG-HRP (horse radish peroxidase) coupled goat polyclonal antibody (1:1000 dilution; Promega, Mannheim, Germany) for 1 h at RT. The cells were washed again two times and stained with 50 μl 3,3′,5,5′-Tetramethylbenzidine (TMB; Seramun Diagnostic GmbH, Dolgenbrodt, Germany) solution for 10 to 20 min at RT. Finally, TMB was removed and the foci visualized by microscopy. The titer was calculated using the Spearman-Kaerber formula (10).

1.3. Infection of AGR129 Mice and Virus Extraction from Ovaries

Six to ten week old female mice were inoculated intraperitoneally with 1×10⁷ TCID₅₀ of virus in 100 μl and controlled daily for signs of disease. Normally mice that survived for 100 days were sacrificed, although in some specific experiments mice were monitored for longer. Mice that showed a hunched position, or had difficulties in moving were sacrificed and the virus isolated from the ovaries. The ovaries were removed and macerated in 100 μl of cold PBS in tubes on ice using a pestle. The tubes were filled up to 1 ml with PBS and frozen at −80° C. until viral titration.

1.4. In Vitro and In Vivo Attenuation Profiling In Vitro Attenuation Profile of Viral Stocks CVA, MVA-I721, MVA-572 and MVA-BN

First the attenuation of the different viruses was tested in human and mouse cell lines and those results were compared to the attenuation in CEF cells. Six-well plates (BD Biosciences, Heidelberg Germany) were seeded with the appropriate cell lines and incubated overnight at 37° C., 5% CO₂ to obtain 80-90% confluence (1×10⁶/well). Following incubation, medium was removed and cells were infected with 500 μl of the different viruses to obtain a multiplicity of infection (m.o.i.) of 0.05. Following incubation for 1 h at 37° C., 5% CO₂, the viral inocula were removed by gentle aspiration, 2 ml DMEM containing 2% FCS was added to each well and plates were incubated for a further 4 days at 37° C., 5% CO₂. Triplicate wells for each virus and appropriate mock infected controls were used. After 4 days, the cells were scraped directly into the medium and harvested. Harvests were then freeze/thawed 3 times to isolate the virus, which were then used in titration experiments. The results were expressed as the geometric ratio (output versus input after 4 days) together with the standard error of the mean. The cells or cell lines were characterized as being either permissive, semi-permissive or non-permissive based on a virus replication ratio of >25-fold, 1-25-fold or less than 1-fold respectively (5).

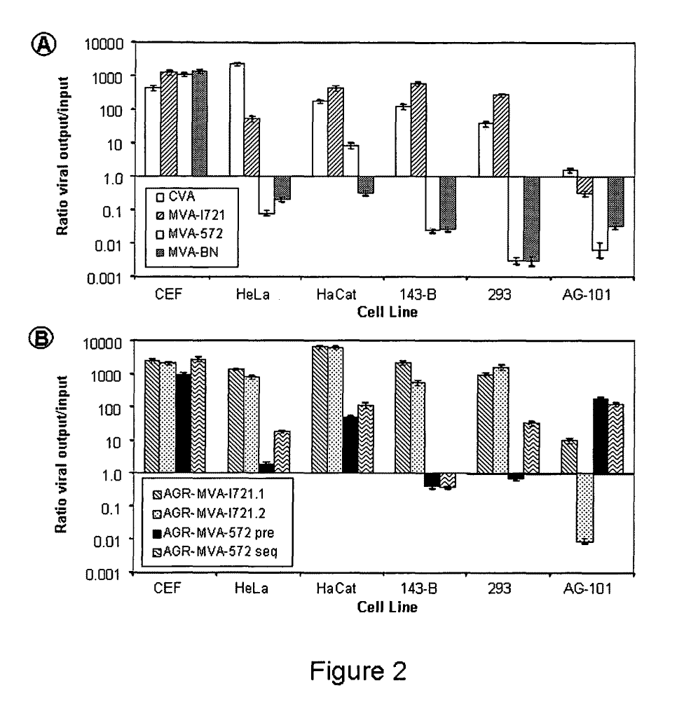

To investigate whether the three MVA viruses shared the same growth properties, their ability to replicate in primary CEF cells and several human cell lines was evaluated and also compared to the parental vaccinia strain CVA. As illustrated in FIG. 2, panel A CEF cells and all human cell lines were permissive for CVA. Interestingly, all three MVA viruses had an increased ability to replicate on CEF cells compared to CVA with geometric mean ratios >2.5-fold higher. Moreover, there were clear differences in the ability of the three MVA viruses to replicate in the human cell lines. MVA-BN was clearly shown to be the most attenuated virus and failed to replicate in any of the human cell lines tested. In contrast, all human cell lines were permissive for MVA-I721 and this MVA virus actually had a higher replication in HaCat, 143-B and 293 cells than CVA. Even MVA-572, which was used in Germany during the smallpox eradication program, replicated in the human HaCat cell line, which was shown to be semi-permissive for this MVA virus. The only virus that replicated in the murine cell line AG-101 was CVA, although this was only limited with a geometric mean ratio of 1.5.

### In Vivo Attenuation Profile of MVA Viruses in Immune-Deficient Mice

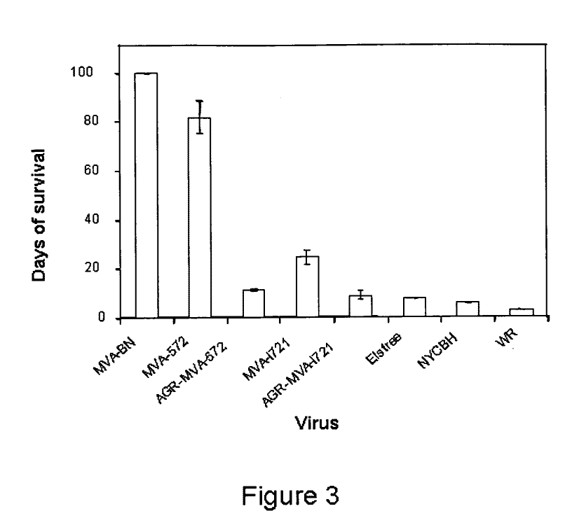

Since MVA has been shown to be safe in immune suppressed animals (11, 12), the safety of the three MVA viruses was tested in the severely immune compromised mouse strain, AGR129 (9, 24). Through gene deletions, the AGR129 mice have no ability to bind IFN type I or II; generate mature T and B cells and also have non-functional innate immune cells, such as NK and macrophages (24). The combined absence of these crucial immune elements renders the AGR129 mice extremely immune compromised and creates an ideal animal model to evaluate the safety of vaccinia viruses. As shown in FIG. 3, mice inoculated with vaccinia virus strain Elstree or NYCBH (1×10⁷ TCID₅₀) died within 8 to 6 days respectively, while mice inoculated with vaccinia virus strain WR survived less than 72 h. Surprisingly, mice inoculated with MVA-I721 (10⁷ TCID₅₀) died within 25 days, while mice infected with the same dose of MVA-572 died within an average time of 82 days. In contrast, AGR129 mice inoculated with MVA-BN survived for more than 100 days and as long as 180 days before the animals were sacrificed. At no time point could MVA virus be isolated from the AGR129 mice inoculated with MVA-BN, although viral titers >1×10⁷ TCID₅₀/ml could be isolated from the ovaries of the mice inoculated with MVA-1721, MVA-572 or the various vaccinia virus strains (data not shown). The MVA viruses isolated from the dead AGR129 mice were renamed with the AGR prefix (AGR-MVA-572 or AGR-MVA-I721) and used to re-inoculate AGR129 mice. Mice inoculated with AGR-MVA-I721.1 or AGR-MVA-572 died within 9 and 11 days, which is 3 to 7 times faster than mice inoculated with the parental MVA strains MVA-I721 or MVA-572 respectively (FIG. 3).

In Vitro Attenuation Profile of Viral Variants Isolated from AGR129 Mice Inoculated with MVA-I721 and MVA-572

To further determine the basis for the differences for the varying attenuation profiles of the MVA viruses, two MVA viruses from two separate AGR129 mice inoculated with MVA-I721 and called AGR-MVA-I721.1 and AGR-MVA-I721.2 were isolated. Similarly, an MVA virus was isolated from the AGR129 mice inoculated with MVA-572, although in this case the virus was plaque purified by three rounds of limiting dilution and called AGR-MVA-572pre. This MVA virus was further plaque purified by another three rounds of limiting dilution and the isolate was called AGR-MVA-572seq. As illustrated in FIG. 2, panel B, all the AGR-MVA viruses replicated equally well on CEF cells. The two AGR-MVA-I721 viruses had in general a 10-fold increased capacity to replicate in the human cell lines compared to MVA-I721. However, an unexpected finding was that there were differences between the two AGR-MVA-I721 viruses. AGR-MVA-I721.1 replicated in the murine AG-101 cell line, while the second virus (AGR-MVA-I721.2) had a similar phenotype to MVA-I721 and failed to replicate in this murine dendritic cell line. Differences between the AGR viruses was even more surprising when comparing the two AGR-MVA-572 viruses, which represented different plaque purified isolates from the same MVA virus (AGR-MVA-572). The two AGR-MVA-572 viruses differed from MVA-572 and now replicated in the human cell line HeLa and had an increased ability to replicate in HaCat cells. However, AGR-MVA-572seq had an almost 10-fold increased ability to replicate in HeLa cells compared to the other plaque purified virus AGR-MVA-572pre and also replicated in the human 293 cell line, which was non-permissive for AGR-MVA-572pre and MVA-572.

In summary, the assays of the invention provided several surprising results. It is widely accepted that MVA is safe in immune suppressed animals (11, 12) and fails to replicate, or only has a limited replication, in human cells (5, 12, 14, 21). Therefore, it was a surprising finding that not only were all the human cell lines tested permissive for MVA-1721, but that this MVA actually had a 2-7 fold increased ability to replicate on HaCat, 143B and 293 cells compared to CVA, questioning whether MVA-I721 has been attenuated compared to CVA at all. Indeed, while MVA-I721 has created by passaging an MVA strain (MVA-570, 11) in CEF cells, this virus clearly differed from the other two MVA strains evaluated. MVA-572 clearly had a more attenuated profile and only replicated in the HaCat cell line and took 4 times as long as MVA-I721 to kill the AGR129 mice. Similarly, MVA-BN that was derived from MVA-572, by additional passaging and limiting dilution, failed to replicate in any of the cell lines or in immune suppressed mice and represented an altered safer phenotype compared to MVA-572 and MVA-I721.

1.5. Deletion Profiling and Analysis of Viral Genomic DNA by PCR

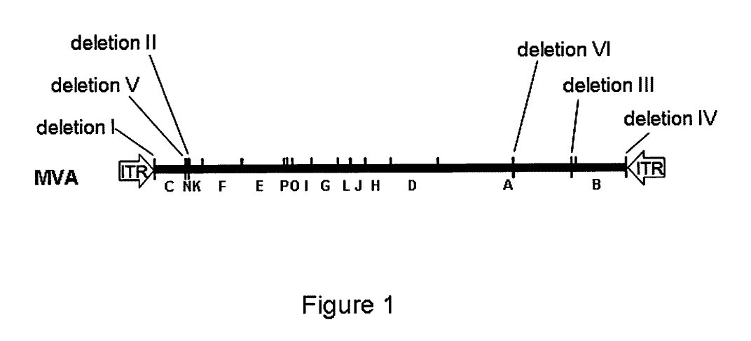

The surprising results described above, prompted the development of an assay that could be used for first-round screening of the attenuation profile of various viruses. To this end, the DNA of multiple viruses cultures with and without prior amplification of the viral population was characterized. DNA was extracted both from cell cultures and virally-infected mouse organs using Blood Quick Pure Kit (Macherey & Nagel, Düren, Germany). PCR analysis was performed to investigate the six deletions within the MVA genome depicted in FIG. 1. A series of primers outside of the deletion sites was designed to amplify the whole deletion site or within the deletion to bind to the CVA locus, as shown in Table 1. The DNA regions of interest were amplified with specific primers (Table 1) for 30 cycles using optimal conditions for each primer pair. The amplified product was analyzed on 0.8% agarose gels.

TABLE 1
PCR primers used to profile deletions in MVA and genomic origin in CVA

PCR product size

| | | | |
|---|---|---|---|
| Primer | Sequence | MVA-BN | CVA |

| | | | | |
|---|---|---|---|---|
| Deletion I | 5′ | 5′-TAA CTT ATA CAG TAC GTA GTA GTA G-3′ | 220 bp | 3634 bp |
| | | (SEQ ID NO: 1) | | |
| | 3′ | 5′-ATG GAT ATC TTT AAA GAA CTA ATC GTA AAA C-3′ | | |
| | | (SEQ ID NO: 2) | | |
| CVA | 5′ | 5′-GCG GTT TTC ATG GAG TCA TTT CTG-3′ | negative | 2848 bp |
| | | SEQ ID NO: 3) | | |
| | 3′ | 5′-GTA TGA TCA TTT TAG ATA ACG ATT GAT-3′ | | |
| | | (SEQ ID NO: 4) | | |
| Deletion II | 5′ | 5′-CTA TAG GTG CGT TGT ATA CAC ATA TTG A-3′ | 400 bp | 3161 bp |
| | | (SEQ ID NO: 5) | | |
| | 3′ | 5′-CAA AGA TGC ATT TAA GGC GGA TGT CCA T-3′ | | |
| | | (SEQ ID NO: 6) | | |
| CVA | 5′ | 5′-TTC GTA AGA TAC TCC TTC ATG AAC-3′ | negative | 2741 bp |
| | | (SEQ ID NO: 7) | | |
| | 3′ | 5′-TGA TGA CAA GGG AAA CAC TGC-3′ | | |
| | | (SEQ ID NO: 8) | | |
| Deletion | 5′ | 5′-GCT GAT AAT AGA ACT TAC GCA AAT ATT A-3′ | 509 bp | 4056 bp |
| III | | (SEQ ID NO: 9) | | |
| | 3′ | 5′-TTA GCA GCT AAA AGA ATA ATG GAA TTG G-3′ | | |
| | | (SEQ ID NO: 10) | | |
| CVA | 5′ | 5′-ATT TAA TAA GAA ATC GAG ACT ACA TTC C-3′ | negative | 3545 bp |
| | | (SEQ ID NO: 11) | | |
| | 3′ | 5′-CTT TAG AAA ATC ATT CGT GTA CTG TG-3′ | | |
| | | (SEQ ID NO: 12) | | |
| Deletion IV | 5′ | 5′-CTA GGT ATT TGT ATC TCA CCG ATA GAG A-3′ | 204 bp | 6659 bp |
| | | (SEQ ID NO: 13) | | |
| | 3′ | 5′-TGT TGG TAG TTC TTC CGT GGA ATC AAT A-3′ | | |
| | | (SEQ ID NO: 14) | | |
| CVA | 5′ | 5′-AGT ACT TTT ATA ATT ATA GAT CAG TCA ACG-3′ | negative | 6450 bp |
| | | (SEQ ID NO: 15) | | |
| | 3′ | 5′-TAA CAC CCT CAG CTA TAT CTG-3′ | | |
| | | (SEQ ID NO: 16) | | |
| Deletion V | 5′ | 5′-GTT GGA TGA ATA GTA TGT CTT AAT AAT-3′ | 329 bp | 5055 bp |
| | | (SEQ ID NO: 17) | | |
| | 3′ | 5′-ACA TTG ATT AAG AAC ATG AGA ATG ACG-3′ | | |
| | | (SEQ ID NO: 18) | | |
| CVA | 5′ | 5′-TGA GTT CAG AAT ATG TTA TAA ATT TAA ATC G-3′ | negative | 4808 bp |
| | | (SEQ ID NO: 19) | | |
| | 3′ | 5′-AGT CAT TCA CCA TAC TCT TTA GG-3′ | | |
| | | (SEQ ID NO: 20) | | |
| Deletion VI | 5′ | 5′-GAT GGT GTC ACA TCA CTA ATC G-3′ | 1398 bp | 5201 bp |
| | | (SEQ ID NO: 21) | | |
| | 3′ | 5′-TGA AAC TCT AAG AGC GGC TAT GAT-3′ | | |
| | | (SEQ ID NO: 22) | | |
| CVA | 5′ | 5′-TCT CTA TCG AGT TTA TCA GAG GC-3′ | negative | 3769 bp |
| | | (SEQ ID NO: 23) | | |
| | 3′ | 5′-AAC GAT AGT ACT GAT GTT CAA CG-3′ | | |
| | | (SEQ ID NO: 24) | | |

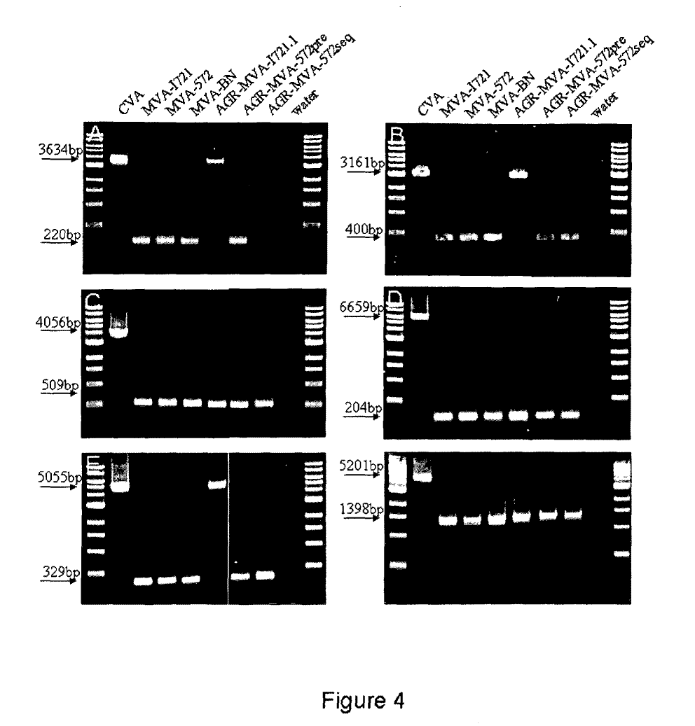

MVA is characterized by having six deletions within the genome compared to CVA (2, 3, 14). By PCR all six deletions described for MVA were present in MVA-I721, MVA-572 and MVA-BN, while as it was anticipated these deletions were absent in CVA (FIG. 4, Table 2). Similarly, all six deletions were detected for both AGR-MVA-572pre as indicated by the correct sized PCR products. Deletion sites II, III, IV, V and VI were illustrated for AGR-MVA-572seq, although no PCR product for deletion site I could be detected (see sequencing below). While deletion sites Ill, IV and VI were also demonstrated for AGR-MVA-I721.1, there appeared to be a mixture for the other three deletion sites (I, II and V) with PCR products detected for both the deletion site (as was anticipated for MVA) and also for the CVA product, indicating the absence of a deletion site (FIG. 4, Table 2 “Deletion MVA” column). Similar results were also found for AGR-MVA-I721.2 (data not shown).

TABLE 2
Summary of the genetic analysis of various MVA viruses by PCR

| | | | | | | | CVA locus | | | | | |
|---|---|---|---|---|---|---|---|---|---|---|---|---|
| | Deletion MVA | | | | | | at MVA deletion | | | | | |
| Virus | I | II | III | IV | V | VI | I | II | III | IV | V | VI |
| CVA | − | − | − | − | − | − | + | + | + | + | + | + |
| MVA-I721 | + | + | + | + | + | + | − | + | − | − | + | − |
| MVA-572 | + | + | + | + | + | + | − | − | − | − | − | − |
| MVA-BN | + | + | + | + | + | + | − | − | − | − | − | − |
| AGR-MVA- | +/− | +/− | + | + | +/− | + | + | + | − | − | + | − |
| I721.1 | | | | | | | | | | | | |
| AGR-MVA- | + | + | + | + | + | + | − | − | − | − | − | − |
| 572pre | | | | | | | | | | | | |
| AGR-MVA- | −* | + | + | + | + | + | − | − | − | − | − | − |
| 572seq | | | | | | | | | | | | |

*Extended deletion I;
“+/−” Presence of both PCR products (MVA-BN deletion and CVA)

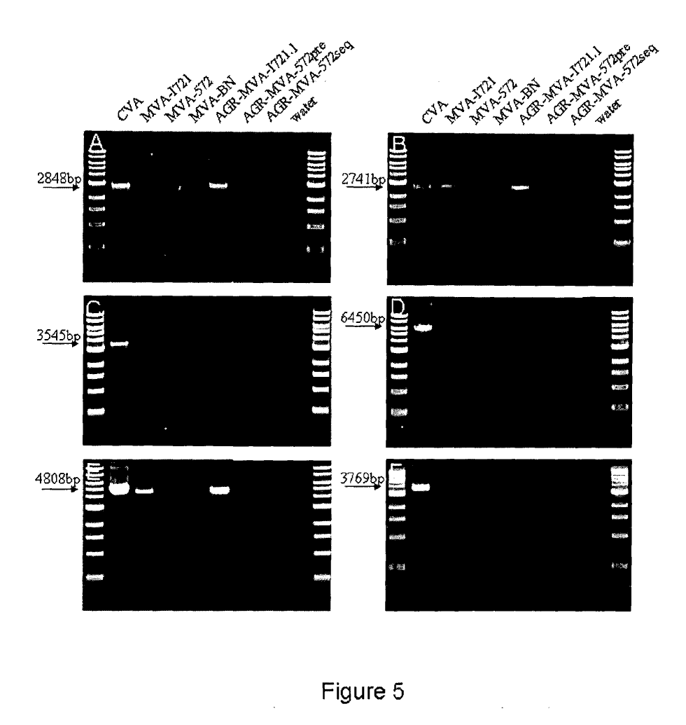

To confirm the absence of deletion sites in AGR-MVA-I721.1, PCR primers were designed from the CVA loci, such that the primers would only bind to genomic DNA if the deletion within the genome (deletion site I-VI) was not present. Indeed, this analysis confirmed similar sized PCR products as amplified from CVA for AGR-MVA-I721.1 at deletion site I, II and V, confirming that AGR-MVA-I721.1 was a polyclonal mixture containing MVA viruses with 3 to 6 deletion sites within the genome (FIG. 5, Table 2 “CVA locus at MVA deletion” column).

To investigate whether any of the parental MVA viruses constituted a polyclonal viral mixture with viruses encoding less than the expected six deletions for MVA, the same PCR analysis was performed on all the MVA viruses (FIG. 5, Table 2). No CVA loci could be detected by PCR at any of the 6 deletion sites in MVA-BN, MVA-572, AGR-MVA-572pre and AGR-MVA-572seq confirming the previous PCR analysis that there appeared to be no variant viruses with less than six deletions expected in MVA. However, CVA loci were amplified from the MVA-I721 genome at deletion site II and V (FIG. 5, Table 2).

One of the reported generalizations about MVA is that they have six deletions within the viral genome compared to vaccinia virus (3, 14). In contrast to these generalizations, and since such differences were found in the phenotype of the MVA, it was posited whether all MVA, particularly MVA-I721, represented an MVA virus or if this method could be used to screen for molecular differences (more sensitive, more rapid assay). However, by PCR using primers flanking the deletions, all three MVA viruses, including MVA-I721, encoded the same six deletions within their genomes. Moreover, sequencing revealed that all three MVA viruses had a 100% identical genome within the coding region and as such could be positively characterized as authentic MVA viruses if one followed the definitions that one of skill in the art would find in the literature (3).

This method resolved the discrepancy between altered phenotypes, but identical genotypes by examining the MVA viruses isolated from the AGR129 mice that died after being inoculated with MVA-I721. Both AGR-MVA-I721.1 and AGR-MVA-I721.2 had an increased ability to replicate in human cells and actually led to the death of AGR129 at a faster rate than the parental MVA virus, MVA-I721. Indeed, these MVA viruses had a similar phenotype in the AGR129 mice as two vaccinia strains considered to have a medium pathogenicity in animals (8). Both these isolates appeared to be polyclonal mixtures of viruses, some of which did not have deletions at sites I, II and/or V. This was confirmed by PCR using primers designed from the CVA regions, which demonstrated that the CVA loci were present. Given that these MVA variants had additional DNA derived from CVA meant that they had to have been enriched from the parental MVA strain in the immune suppressed animals, due to their increased ability to replicate in these mice. Indeed, the absence of deletion sites II and V was confirmed by PCR (using CVA primers) in MVA-I721, clearly indicating that this MVA was a heterogenic mixture made of different MVA viruses. The absence of deletion sites by PCR was not demonstrated for any of the other MVA viruses, including the AGR-MVA-572 variants. Interestingly however, the absence of deletion I was also not demonstrated in MVA-I721 and as MVA variants without deletion I were isolated from the mice inoculated with MVA-I721 this genotype had to have been present within the parental MVA, although presumably at a level below the detection of the PCR.

### Example 2

### Mutation Profiling of Attenuated and Replication-Competent MVA Viruses

2.1. Identification of New MVA Mutation Patters by DNA Sequencing

It was found that AGR-MVA-572pre and AGR-MVA-572seq had the six MVA specific genomic deletions and that no CVA equivalent DNA was present at these loci (FIGS. 4 and 5, Table 2). Therefore, to further identify changes within the genome associated with the changed phenotype compared to the parental MVA virus MVA-572, the sequence of the entire genomes from these AGR viruses were compared to MVA-572, MVA-BN and MVA-I721.

Genomic DNA of the various MVA strains were isolated with a commercially available kit (NucleoSpin® Blood Quick Pure, Macherey-Nagel, Duren, Germany) using 2×10⁷-1×10⁸ TCID₅₀ of viral stock suspensions. Purified viral genomic DNA was used as template to amplify DNA fragments of 5 kB covering the complete coding sequence starting between the repetitive sequences of the ITRs and ORF MVA001L and extending through ORF MVA193R (numbering according to (3) with an overlap of ˜500 base pairs each. Briefly, PCR fragments were amplified using the TripleMaster® PCR system (Eppendorf, Hamburg, Germany) and purified with the QIAquick PCR purification kit (QIAGEN, Hilden, Germany). The PCR fragments were directly sequenced by Sequiserve GmbH (Vaterstetten, Germany) with an Applied Biosystems 3730 DNA Analyzer and Sequencing Analysis software v5.0 using 10-14 custom-designed primers per PCR fragment. Contigs were assembled and analyzed using Vector NTI Advance™ 9.1. The final DNA sequence represents a consensus of at least 3 independent readings per nucleotide. The results are summarized in Table 3.

100% identity was found in coding region between MVA-BN (Genbank accession number DQ983238), MVA-572 (Genbank accession number DQ983237) and MVA-I721 (Genbank accession number DQ983236; data not shown). On the other hand, at least eight nucleotide differences were found in AGR-MVA-572pre (Genbank accession number DQ983239), compared to the other MVA viruses, such as nt 137398: 1A insertion; nt 133176: G/A; 27698: 2 nt deletion; 86576: 1 nt deletion; nt 126375: C/A; nt 135664: G/A; nt 149358: G/T; nt 153212: C/T. For AGR-MVA-572seq (Genbank accession number DQ983240) only three mutations were identified; two that were also present in AGR-MVA-572pre (nt 137398: 1A insertion; nt 133176: G/A), and a unique mutation for this MVA virus (nt 85017: C/T). Moreover, AGR-MVA-572seq had an extended deletion I site with the loss of an additional 13 kb (Δ1L-13 L), which is why no PCR product was identified for deletion I (FIG. 4, Table 2).

Sequencing revealed that a number of point mutations could be identified in the two AGR-MVA-572 plaque purified clones compared to MVA-572. These mutations can either have resulted from adaptation of the MVA viruses in the AGR129 mice, resulting in an improved ability to replicate in the host, or as with the AGR-MVA-I721 variants, an enrichment of viral populations from within the parental MVA strain. Given that no mutations or adaptations occurred in more than 50 AGR129 mice inoculated with MVA-BN, coupled with the proven enrichment of the AGR-MVA-I721 variants from MVA-I721, it was concluded from this new screening/profiling method that MVA-572 is also a heterogenic mixture made up of MVA variants with an altered genotype and phenotype that can be enriched using AGR129 mice.

TABLE 3
Mutations found in AGR-MVA-572-pre and AGR-MVA-572-seq compared to MVA-BN

| | | Gene(s) affected by mutation^{&} | | | |
|---|---|---|---|---|---|
| | | AGR-MVA-572-pre | | AGR-MVA-572-seq | |
| | Position of mutation^{§} | nt exchange | aa exchange | nt exchange | aa exchange |
| 001L-016L | length of | no deletion | — | ~13 kbp | ORFs 005R, |
| (extension | deletion: ~13 kbp | | | deleted | 006L, 007R, 008L |
| of deletion I) | 3′ end of | | | | deleted |
| | deletion^{$}: 17513 | | | | |
| 094L (RNA | 85017 | — | — | C > T | Ala > Val |
| pol cofactor) | | | | | |
| 148R | 137398-404 | 1A inserted | frameshift | 1A deleted | frameshift |
| 142R | 133176 | G > A | Gly > Asp | G > A | Gly > Asp |
| 031L (kelch- | 27698/99 | 2nt deletion | frameshift | — | — |
| like protein) | | (TA) | | | |
| IGR^{#} | 86576 | 1nt deletion | silent | — | — |
| 094L/095R | | (T) | | | |
| 135R (RNA pol | 126375 | C > A | Ala > Asp | — | — |
| subunit rpo132) | | | | | |
| 146R | 135664 | G > A | Ala > Thr | — | — |
| IGR^{#} | 149358 | G > T | silent | — | — |
| 164R/165R | | | | | |
| 170R | 153212 | C > T | Thr > Met | — | — |

| | | | |
|---|---|---|---|
| AGR mouse | not applicable | 9-10 | 9-10 |
| pathogenicity | | (n = 3) | (n = 3) |
| (days to death) | | | |

^{&}gene names according to Antoine et al., 1998. Virology 244, 365-396.
^{§}numbering refers to nucleotide sequence of strain “modified vaccinia Ankara” (MVA), GenBank accession no. U94848 (Antoine et al., 1998. Virology 244, 365-396.)
^{$}5′ end of deletion appears to be variable
^{#}IGR = intergenic region
— = not present

Most of the mutations that were identified in the two AGR-MVA-572 strains affect genes such as 148R, 142R, 146R with reportedly unknown functions (3). Thus, the potential role of these mutations in the adaptation of MVA to the murine host remains unclear at present, although is likely further complicated by the fact that both these MVA variants can represent polyclonal mixtures with different genotypes. However, this assay implicates these mutations in the pathogenicity (i.e., replication and/or attenuation potential) of MVA. Thus, any of the methods and PCR primers described above can be used as part of kits and assays for the screening and profiling of the pathogenicity of MVA virus populations.

### REFERENCES

1. Altenburger, J. 1993. U.S. Pat. No. 5,185,146.
2. Altenburger, W., C. P. Suter, and J. Altenburger. 1989. Partial deletion of the human host range gene in the attenuated vaccinia virus MVA. Arch Virol 105:15-27.
3. Antoine, G., F. Scheiflinger, F. Dorner, and F. G. Falkner. 1998. The complete genomic sequence of the modified vaccinia Ankara strain: comparison with other orthopoxviruses. Virology 244:365-96.
4. Blanchard, T. J., A. Alcami, P. Andrea, and G. L. Smith. 1998. Modified vaccinia virus Ankara undergoes limited replication in human cells and lacks several immunomodulatory proteins: implications for use as a human vaccine. J Gen Virol 79 (Pt 5):1159-67.
5. Carroll, M. W, and B. Moss. 1997. Host range and cytopathogenicity of the highly attenuated MVA strain of vaccinia virus: propagation and generation of recombinant viruses in a nonhuman mammalian cell line. Virology 238:198-211.
6. Chaplin, P., P. Howley, and C. Meisinger. 2005. U.S. Pat. No. 6,913,752 B2.
7. Drexler, I., K. Heller, B. Wahren, V. Erfle, and G. Sutter. 1998. Highly attenuated modified vaccinia virus Ankara replicates in baby hamster kidney cells, a potential host for virus propagation, but not in various human transformed and primary cells. J Gen Virol 79 (Pt 2):347-52.
8. Esposito, J. J., and F. Fenner. 1999. Poxviruses. In: Fields, Virology, Third Edition Ed: Fields, B. N., Knipe, D. M., Howley, et al.
9. Grob, P., V. E. Schijns, M. F. Van den Broek, S. P. Cox, M. Ackermann, and M. Suter. 1999. Role of the individual interferon systems and specific immunity in mice in controlling systemic dissemination of attenuated pseudorabies virus infection. J Virol 73:4748-54.
10. Kaerber, G. 1931. Beitrag zur kollektiven Behandlung pharmakologischer Reihenversuche. Arch Expt Pathol Pharmakol 162:480-82.
11. Mayr, A., v. Hochstein-Mintzel, and H. Stickel. 1975. Abstammung, Eigenschaften und Verwendung des attenuierten Vaccinia-Stammes MVA. Infection 3:6-14.
12. Mayr, A., and E. Munz. 1964. [Changes in the vaccinia virus through continuing passages in chick embryo fibroblast cultures]. Zentralbl Bakteriol [Orig] 195:24-35.
13. Mayr, A., H. Stickl, H. K. Muller, K. Danner, and H. Singer. 1978.
[The smallpox vaccination strain MVA: marker, genetic structure, experience gained with the parenteral vaccination and behavior in organisms with a debilitated defence mechanism (author's transl)]. Zentralbl Bakteriol [B] 167:375-90.
14. Meyer, H., G. Sutter, and A. Mayr. 1991. Mapping of deletions in the genome of the highly attenuated vaccinia virus MVA and their influence on virulence. J Gen Virol 72:1031-8.
15. Moss, B., M. W. Carroll, L. S. Wyatt, J. R. Bennink, V. M. Hirsch, S. Goldstein, W. R. Elkins, T. R. Fuerst, J. D. Lifson, M. Piatak, N. P. Restifo, W. Overwijk, R. Chamberlain, S. A. Rosenberg, and G. Sutter. 1996. Host range restricted, non-replicating vaccinia virus vectors as vaccine candidates. Adv Exp Med Biol 397:7-13.
16. Nunez, R., P. Grob, S. Baumann, A. Zuniga, M. Ackermann, and M. Suter. 1999. Immortalized cell lines derived from mice lacking both type I and type II IFN receptors unify some functions of immature and mature dendritic cells [In Process Citation]. Immunol Cell Biol 77:153-63.
17. Spehner, D., R. Drillien, F. Proamer, C. Houssais-Pecheur, M. A. Zanta, M. Geist, K. Dott, and J. M. Balloul. 2000. Enveloped virus is the major virus form produced during productive infection with the modified vaccinia virus Ankara strain. Virology 273:9-15.
18. Stickl, H., V. Hochstein-Mintzel, and H. C. Huber. 1973. [Primary vaccination against smallpox after preliminary vaccination with the attenuated vaccinia virus strain MVA and the use of a new “vaccination stamp”]. Munch Med Wochenschr 115:1471-3.
19. Stickl, H., V. Hochstein-Mintzel, A. Mayr, H. C. Huber, H. Schafer, and A. Holzner. 1974. [MVA vaccination against smallpox: clinical tests with an attenuated live vaccinia virus strain (MVA) (author's transl)]. Dtsch Med Wochenschr 99:2386-92.
20. Sutter, G. 1990. Das Genom des Vacciniavirusstammes Ankara und seine Änderung wäthrend der Attenuierung. Doctoral Thesis University of Munich. 3.
21. Sutter, G., and B. Moss. 1992. Nonreplicating vaccinia vector efficiently expresses recombinant genes. Proc Natl Acad Sci USA 89:10847-51.
22. Sutter, G., and C. Staib. 2003. Vaccinia vectors as candidate vaccines: the development of modified vaccinia virus Ankara for antigen delivery. Curr Drug Targets Infect Disord 3:263-71.
23. Vollstedt, S., S. Arnold, C. Schwerdel, M. Franchini, G. Alber, J. P. Di Santo, M. Ackermann, and M. Suter. 2004. Interplay between alpha/beta and gamma interferons with B, T, and natural killer cells in the defense against herpes simplex virus type 1. J Virol 78:3846-50.
24. Vollstedt, S., M. O'Keeffe, B. Odermatt, R. Beat, B. Glanzmann, M. Riesen, K. Shortman, and M. Suter. 2004. Treatment of neonatal mice with Flt3 ligand leads to changes in dendritic cell subpopulations associated with enhanced IL-12 and IFN-alpha production. Eur J Immunol 34:1849-60.
25. Weltzin, R., J. Liu, K. V. Pugachev, G. A. Myers, B. Coughlin, P. S. Blum, R. Nichols, C. Johnson, J. Cruz, J. S. Kennedy, F. A. Ennis, and T. P. Monath. 2003. Clonal vaccinia virus grown in cell culture as a new smallpox vaccine. Nat Med 9:1125-
26. Wyatt, L. S., M. W. Carroll, C. P. Czerny, M. Merchlinsky, J. R. Sisler, and B. Moss. 1998. Marker rescue of the host range restriction defects of modified vaccinia virus Ankara. Virology 251:334-42.

1. A method of screening an MVA nucleic acid sample for mutations comprising:
preparing an MVA nucleic acid sample; and
determining whether the MVA nucleic acid sample includes one or more minority viral genotypes having a different genomic DNA sequence at a site selected from one or more of the following sites:
deletion I site; nt 85017; nts 137398-404; nt 133176; nt 27698; nt 27699; nt 86576; nt 126375; nt 135664; nt 149358; and nt 153212 of an MVA virus strain having at least one of the following properties:
i) capability of reproductive replication in vitro in chicken embryo fibroblasts (CEF) but no capability of reproductive replication in the human keratinocyte cell line (HaCaT), the human embryo kidney cell line (293), the human bone osteosarcoma cell line (143B), and the human cervix adenocarcinoma cell line (HeLa), and
(ii) failure to replicate in a mouse model that is incapable of producing mature B and T cells and as such is severely immune compromised and highly susceptible to a replicating virus.

2. The method of claim 1, wherein said MVA virus strain has both of properties (i) and (ii).

3. The method of claim 1, wherein said MVA virus strain is MVA-BN as deposited at the European Collection of Cell Cultures (ECACC) under number V00083008.

4. The method of claim 1, wherein the nucleic acid sample is analyzed by PCR.

5. The method of claim 1, wherein the MVA nucleic acid sample is prepared from an animal host.

6. The method of claim 5, wherein the animal host is an immunocompromised mouse.

7. A kit for screening an MVA nucleic acid sample for mutations comprising an oligonucleotide primer for amplifying an MVA nucleic acid by PCR,
wherein said primer amplifies a segment of MVA DNA comprising a site selected from one or more of the following sites:
deletion I site; nt 85017; nts 137398-404; nt 133176; nt 27698; nt 27699; nt 86576; nt 126375; nt 135664; nt 149358; and nt 153212 of an MVA virus strain having at least one of the following properties:
i) capability of reproductive replication in vitro in chicken embryo fibroblasts (CEF) but no capability of reproductive replication in the human keratinocyte cell line (HaCaT), the human embryo kidney cell line (293), the human bone osteosarcoma cell line (143B), and the human cervix adenocarcinoma cell line (HeLa), and
(ii) failure to replicate in a mouse model that is incapable of producing mature B and T cells and as such is severely immune compromised and highly susceptible to a replicating virus.

8. The kit of claim 7, wherein said MVA virus strain has both of properties (i) and (ii).

9. The kit of claims 7, wherein said MVA virus strain is MVA-BN as deposited at the European Collection of Cell Cultures (ECACC) under number V00083008.

10. The method of claim 1, wherein the site is deletion I site.

11. The method of claim 1, wherein the site is nt 85017.

12. The method of claim 1, wherein the site is nts 137398-404.

13. The method of claim 1, wherein the site is nt 133176.

14. The method of claim 1, wherein the site is nt 27698.

15. The method of claim 1, wherein the site is nt 27699.

16. The method of claim 1, wherein the site is nt 86576.

17. The method of claim 1, wherein the site is nt 126375.

18. The method of claim 1, wherein the site is nt 135664.

19. The method of claim 1, wherein the site is nt 149358.

20. The method of claim 1, wherein the site is nt 153212.

21. The kit of claim 7, wherein the site is deletion I site.

22. The kit of claim 7, wherein the site is nt 85017.

23. The kit of claim 7, wherein the site is nts 137398-404.

24. The kit of claim 7, wherein the site is nt 133176.

25. The kit of claim 7, wherein the site is nt 27698.

26. The kit of claim 7, wherein the site is nt 27699.

27. The kit of claim 7, wherein the site is nt 86576.

28. The kit of claim 7, wherein the site is nt 126375.

29. The kit of claim 7, wherein the site is nt 135664.

30. The kit of claim 7, wherein the site is nt 149358.

31. The kit of claim 7, wherein the site is nt 153212.


SEQUENCE LISTING (24 sequences)

SEQ ID NO: 1 - DNA, 25 bp, Artificial
  Deletion site I 5′ primer for MVA
taacttatacagtacgtagtagtag

SEQ ID NO: 2 - DNA, 31 bp, Artificial
  Deletion site I 3′ primer for MVA
atggatatctttaaagaactaatcgtaaaac

SEQ ID NO: 3 - DNA, 24 bp, Artificial
  Deletion site I 5′ primer for CVA
gcggttttcatggagtcatttctg

SEQ ID NO: 4 - DNA, 27 bp, Artificial
  Deletion site I 3′ primer for CVA
gtatgatcattttagataacgattgat

SEQ ID NO: 5 - DNA, 28 bp, Artificial
  Deletion site II 5′ primer for MVA
ctataggtgcgttgtatacacatattga

SEQ ID NO: 6 - DNA, 28 bp, Artificial
  Deletion site II 3′ primer for MVA
caaagatgcatttaaggcggatgtccat

SEQ ID NO: 7 - DNA, 24 bp, Artificial
  Deletion site II 5′ primer for CVA
ttcgtaagatactccttcatgaac

SEQ ID NO: 8 - DNA, 21 bp, Artificial
  Deletion site II 3′ primer for CVA
tgatgacaagggaaacactgc

SEQ ID NO: 9 - DNA, 28 bp, Artificial
  Deletion site III 5′ primer for MVA
gctgataatagaacttacgcaaatatta

SEQ ID NO: 10 - DNA, 28 bp, Artificial
  Deletion site III 3′ primer for MVA
ttagcagctaaaagaataatggaattgg

SEQ ID NO: 11 - DNA, 28 bp, Artificial
  Deletion site III 5′ primer for CVA
atttaataagaaatcgagactacattcc

SEQ ID NO: 12 - DNA, 26 bp, Artificial
  Deletion site III 3′ primer for CVA
ctttagaaaatcattcgtgtactgtg

SEQ ID NO: 13 - DNA, 28 bp, Artificial
  Deletion site IV 5′ primer for MVA
ctaggtatttgtatctcaccgatagaga

SEQ ID NO: 14 - DNA, 28 bp, Artificial
  Deletion site IV 3′ primer for MVA
tgttggtagttcttccgtggaatcaata

SEQ ID NO: 15 - DNA, 30 bp, Artificial
  Deletion site IV 5′ primer for CVA
agtacttttataattatagatcagtcaacg

SEQ ID NO: 16 - DNA, 21 bp, Artificial
  Deletion site IV 3′ primer for CVA
taacaccctcagctatatctg

SEQ ID NO: 17 - DNA, 27 bp, Artificial
  Deletion site V 5′ primer for MVA
gttggatgaatagtatgtcttaataat

SEQ ID NO: 18 - DNA, 27 bp, Artificial
  Deletion site V 3′ primer for MVA
acattgattaagaacatgagaatgacg

SEQ ID NO: 19 - DNA, 31 bp, Artificial
  Deletion site V 5′ primer for MVA
tgagttcagaatatgttataaatttaaatcg

SEQ ID NO: 20 - DNA, 23 bp, Artificial
  Deletion site V 3′ primer for CVA
agtcattcaccatactctttagg

SEQ ID NO: 21 - DNA, 22 bp, Artificial
  Deletion site VI 5′ primer for MVA
gatggtgtcacatcactaatcg

SEQ ID NO: 22 - DNA, 24 bp, Artificial
  Deletion site VI 3′ primer for MVA
tgaaactctaagagcggctatgat

SEQ ID NO: 23 - DNA, 23 bp, Artificial
  Deletion site VI 5′ primer for CVA
tctctatcgagtttatcagaggc

SEQ ID NO: 24 - DNA, 23 bp, Artificial
  Deletion site VI 3′ primer for CVA
aacgatagtactgatgttcaacg